(19) United States
(12) Patent Application Publication
(10) Pub. No.: US 2005/0043515 A1
(43) Pub. Date: Feb. 24, 2005

(54) Tobacco-specific nitrosamine detection assays and reagents

(76) Inventors: Michael Craig Brown, North East, MD (US); Dale Vernon Onisk, Bear, DE (US); James W. Stave, Bear, DE (US); Yichun Xu, Newark, DE (US); David M. Johnson, Owensboro, KY (US); Charles H. Melander, Owensboro, KY (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/857,165

(22) Filed: May 28, 2004

Related U.S. Application Data
(60) Provisional application No. 60/474,184, filed on May 29, 2003.

(51) Int. Cl. C12Q 1/68; C07K 16/00

(52) U.S. Cl. 530/387.1

(57) ABSTRACT

Haptens, ligands (including antibodies), immunoassays, and test kits for detecting tobacco-specific nitrosamines (TSNAs) in samples such as tobacco products are provided. The hapten is a molecule that contains one or more structural characteristics of one or more TSNAs, and is bound to an immunogenic carrier. An antibody specific for one or more TSNAs is produced by immunizing an animal with the hapten bound to the carrier. Immunoassays and test kits using those antibodies are also provided.

### CROSS-REFERENCE TO RELATED APPLICATION

This application claims benefit of U.S. Provisional Patent Application No. 60/474,184 filed May 29, 2003.

### FIELD OF THE INVENTION

The present invention relates to the field of diagnostic testing and more specifically relates to assays and reagents for the detection of tobacco-specific nitrosamines.

### BACKGROUND OF THE INVENTION

Tobacco-specific nitrosamines (TSNAs) are chemicals formed by the nitrosation of secondary and tertiary amines of tobacco alkaloids including nicotine, nornicotine, anatabine, and anabasine. TSNAs are found in some tobacco and tobacco products. At least eight different TSNAs are known. The four principal TSNAs, those typically found to be present in the highest concentrations, are N-nitrosonicotine (NNN), 4-(methylnitrosamino)-1-(3-pyridyl)-1-butanone (NNK), N-nitrosoanabasine (NAB) and N-nitrosoanatabine (NAT). Minor compounds, those typically found at significantly lower levels than the principal TSNAs, include 4-(methylnitrosamino)4-(3-pyridyl)butanal (NNA), 4-(methylnitrosamino)-1-(3-pyridyl)-1-butanol (NNAL), 4-(methylnitrosamino)4-(3-pyridyl)-1-butanol (iso-NNAL), and 4-(methylnitrosamino)-4-(3-pyridyl)-1-butyric acid (iso-NNAC).

Scientists, technicians, and other laboratory workers desire the ability to detect the presence or quantity of TSNAs in a variety of substances such as tobacco, tobacco products, and tobacco preparations. There is interest in quantifying both the levels of individual TSNA compounds and the total TSNA concentration, which represents the sum of the levels of all TSNAs in a substance being evaluated.

The presently available methods used for quantifying TSNAs are both complicated and costly. Samples such as tobacco are ground, extracted with methylene chloride or alkaline aqueous solutions, and subjected to extensive differential extraction. The final sample is then analyzed by gas chromatography using a thermal energy analyzer for detection (GC-TEA). These currently accepted methods are expensive, often resulting in costs ranging from $30 to $300 per sample. Additionally, such tests are time consuming, with a single analyst typically having the capacity to process only 20 samples per eight hour shift. While robotic sample preparation methods exist, the cost is prohibitive. Furthermore, TSNA sample preparation generates vast quantities of waste solvent, the disposal of which is also costly. Therefore, inexpensive TSNA tests that are fast and easy to perform are in high demand.

Binding assays, such as immunoassays, offer a reasonable approach as an alternative to slower, more expensive reference methods frequently encountered in environmental, human, veterinary, and agricultural diagnostics. Such assays offer speed and sensitivity, and have been found to be useful, field-capable analytical tools. The key component of an immunoassay is the antibody or antibodies employed. Such antibodies should have sufficient affinity to be able to detect the compounds of interest in the sample and sufficient specificity to recognize only the compounds of interest and not closely related compounds.

It is known by those skilled in the art that TSNAs are structurally similar to the nicotine compounds from which they are typically derived. The structure of nicotine and several nicotine derivatives, including TSNAs, are set forth in FIG. 1. In addition, many tobacco preparations contain a combination of nicotine, nicotine derivatives and TSNAs, making analysis difficult. For example, individual TSNAs typically range from 0.5 ppm (0.5 micrograms per gram of dry tobacco) to 20 ppm or higher in tobacco, while nicotine is present at levels as high as 2% by dry weight in tobacco (20,000 ppm). This can represent a molar ratio of 48,000:1 (nicotine to individual TSNAs) in the tobacco. Thus an antibody having extraordinarily low crossreactivity to nicotine is especially desirable in assays for individual or collective TSNAs. If antibodies are to be used in immunoassays to measure the levels of individual TSNAs, it is also desirable that they have very low crossreactivity with other TSNAs that may be present in the samples.

An important step in the development of an immunoassay is the availability or production of antibodies that bind to the analyte to be detected. Small chemical molecules such as TSNAs are often too small to produce an appropriate immune response when injected into laboratory animals. Several attempts have been made in the past to generate antibodies to nicotine and its derivatives. Talbot et al. (Arch. Toxicol. (1990) 64:360-364) described antibodies to the pyridyl oxybutyl moeity of NNK. These antibodies were not specific for NNK in that they were also immunoreactive with related compounds lacking nitroso groups. These antibodies were used for determining proteins alkylated by NNK via the nitroso group, not for directly measuring NNK. Langone et al. (Arch. Biochem. Biophys. (1974), 164:536-543) described similar antibodies.

Other than these two references, the use of immunoassays in connection with tobacco has been largely restricted to measurement of nicotine and its principal metabolite (cotinine) in humans or to immunoassays for tobacco diseases (i.e. tobacco mosaic virus).

What is needed in the art is a binding assay for detecting the presence and amount of TSNAs, preferably a portable binding assay whose results can be interpreted visually without the use of scientific instrumentation.

### SUMMARY OF THE INVENTION

The present invention overcomes the problems of the prior art by providing reagents and methods such as assays for the detection and quantification of TSNAs. The reagents and assays described herein contain ligands (such as antibodies) having both affinity and specificity for TSNAs, including, but not limited to, antibodies having specificity for all TSNAs, and antibodies having specificity for one or more TSNAs while lacking significant crossreactivity for other TSNAs. Preferably, the antibodies have minimal crossreactivity with nicotine or nicotine derivatives or metabolites. The reagents also include haptens that can be used in producing the antibodies provided herein, as well as methods for producing such antibodies. Immunoassays for detecting and/or quantifying TSNAs as well as kits for performing such immunoassays are also described herein.

The preferred antibody is produced by immunizing an animal with a hapten composed of a TSNA molecule linked to an immunogenic carrier. The preferred hapten is structurally similar to one or more TSNAs and is preferably conjugated to the carrier through a linker group and a spacer group. Immunization of an animal with the immunogen results in the production of antibodies specific for one or more TSNAs. Suitable immunogenic carriers include proteins, polypeptides, peptides, polysaccharides, carbohydrates, polymers, and solid phase substances.

The ligands are optionally assembled in a kit with conventional assay reagents for the detection of one or more TSNAs in a sample. A preferred kit contains either monoclonal antibodies, polyclonal antibodies, or both, and optionally includes a standard for determining the presence or relative concentration of one or more TSNAs in the sample.

It is therefore an object of the present invention to provide ligands (such as antibodies), analytes, immunogens, assay methods, and kits for the detection of TSNAs in a sample, preferably a sample containing tobacco or a tobacco product or preparation.

It is a further object of the present invention to provide immunogens that, upon administration to an animal, will elicit production of antibodies having binding affinity for one or more TSNAs.

It is a further object of the present invention to provide haptens useful in making the immunogens of the present invention

It is a further object of the present invention to provide methods of making the antibodies of the present invention.

It is a further object of the present invention to provide a highly sensitive immunoassay for TSNAs.

It is a further object of the present invention to provide a highly sensitive immunoassay for TSNAs that has minimal or nonexistent crossreactivity with nicotine.

It is a further object of the present invention to provide a highly sensitive immunoassay for determining combined concentrations of TSNAs, including, but not limited to, total TSNAs.

It is a further object of the present invention to provide a highly sensitive immunoassay for one or more TSNAs that has minimal or nonexistent crossreactivity with one or more other TSNAs.

These and other objects of the present invention will become apparent after reading the following detailed description of the disclosed embodiments and the appended claims.

### BRIEF DESCRIPTION OF THE DRAWINGS

FIG. 1 is a group of chemical drawings showing the structure of nicotine and several examples of nicotine derivatives, including TSNAs.

FIG. 2 is a group of chemical drawings showing the structure of several examples of haptens described herein.

FIG. 3 is an additional group of chemical drawings showing the structure of several examples of haptens described herein.

FIG. 4 is a diagram depicting examples of methods for synthesis of haptens described herein. In scheme 6 “BOC” refers to a tert Butoxycarbonyl moiety.

FIG. 5 is a diagram depicting examples of immunogens as described herein.

FIG. 6 is a graph depicting the results of several runs of a competitive ELISA immunoassay test of the present invention using nicotine and various TSNAs. The x-axis depicts concentrations of each analyte in micrograms per milliliter. The y-axis depicts B/Bo. The plot containing points illustrated as open diamonds shows values for nicotine. The plot containing points illustrated as solid rectangles or squares shows values for NNK. The plot containing points illustrated as triangles shows values for NNAL. The plot containing points illustrated as solid diamonds shows values for NAT. The plot containing points illustrated as open rectangles or squares shows values for NAB. The plot containing points illustrated as solid circles shows values for NNA. The plot containing points illustrated as open circles shows values for NNN.

FIG. 7 is a graph depicting the results of several runs of a competitive ELISA immunoassay test of the present invention using nicotine and various TSNAs. The x-axis depicts concentrations of each analyte in micrograms per milliliter. The y-axis depicts B/Bo. The plot containing points illustrated as open diamonds shows values for nicotine. The plot containing points illustrated as solid rectangles or squares shows values for NNK. The plot containing points illustrated as open circles shows values for NNN. The plot containing points illustrated as open rectangles or squares shows values for NAB. The plot containing points illustrated as solid diamonds shows values for NAT.

### DETAILED DESCRIPTION OF THE PREFERRED EMBODIMENTS

TSNA haptens, immunogens, ligands (including, but not limited to antibodies) having specificity for the TSNAs, methods for producing ligands, assays for the detection of TSNAs, and kits for performing such assays are provided. In a preferred embodiment, the hapten is a derivatized TSNA molecule and the immunogen is the derivatized TSNA molecule coupled to an immunogenic carrier. A preferred ligand is an antibody having specificity and affinity for the preferred analyte described herein, namely one or more TSNAs. In some embodiments, the antibody has a high affinity for TSNAs but has a minimal, low, or nonexistent crossreactivity with nicotine. In other embodiments, the antibody has a high affinity for several different TSNAs. In other embodiments, the antibody has a high affinity for one or more TSNAs and has a minimal, low, or nonexistent crossreactivity with one or more other TSNAs.

Preferably, the antibodies described herein are produced by the administration of a conjugate, containing derivatized TSNAs bound to an immunogenic carrier, to an animal under conditions effective to induce an antigenic response. The antibodies are subsequently isolated from a biological fluid of the animal. The antibodies described herein include monoclonal and polyclonal antibodies and are useful in immunoassay methods for the detection and quantification of TSNAs in various compositions, including tobacco and tobacco products and preparations.

Definitions

The terms “a”, “an” and “the” as used herein are defined to mean “one or more” and include the plural unless the context is inappropriate.

The term “analyte” refers to any molecule or fragment thereof to be detected or measured including, but not limited to, one or more TSNA chemicals, compounds, or molecules or fragments thereof to be detected or measured.

The term “tobacco-specific nitrosamine” or “TSNA” refers to compounds formed by the nitrosation of secondary and tertiary amines of alkaloids that are naturally present in tobacco, such alkaloids including, but not limited to, nicotine, nornicotine, anatabine, and anabasine. Exemplary TSNA include, but are not limited to, N-nitrosonicotine (NNN), 4-(methylnitrosamino)-1-(3-pyridyl)-1-butanone (NNK), N-nitrosoanabasine (NAB) and N-nitrosoanatabine (NAT), 4-(methylnitrosamino)-4-(3-pyridyl)butanal (NNA), 4-(methylnitrosamino)-1-(3-pyridyl)-1-butanol (NNAL), 4-(methylnitrosamino)4-(3-pyridyl)-1-butanol (iso-NNAL), and 4-(methylnitrosamino)4-(3-pyridyl)-1-butyric acid (iso-NNAC).

The term “ligand” as used herein refers to a molecule that binds to an epitope or binding site. The term, as used herein, includes antibodies, proteins, peptides, polypeptides, amino acids, nucleic acids, carbohydrates, sugars, lipids, organic molecules, polymers, putative therapeutic agents, and the like.

Immunogens

Immunogens useful for producing antibodies to tobacco-specific nitrosamines (TSNAs) contain a hapten conjugated to an immunogenic carrier.

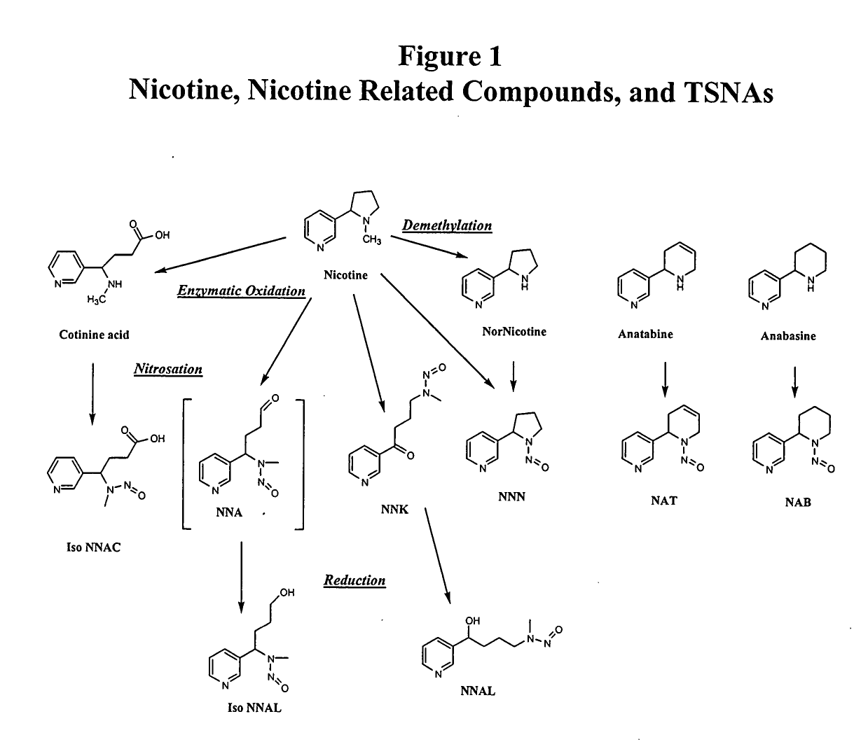

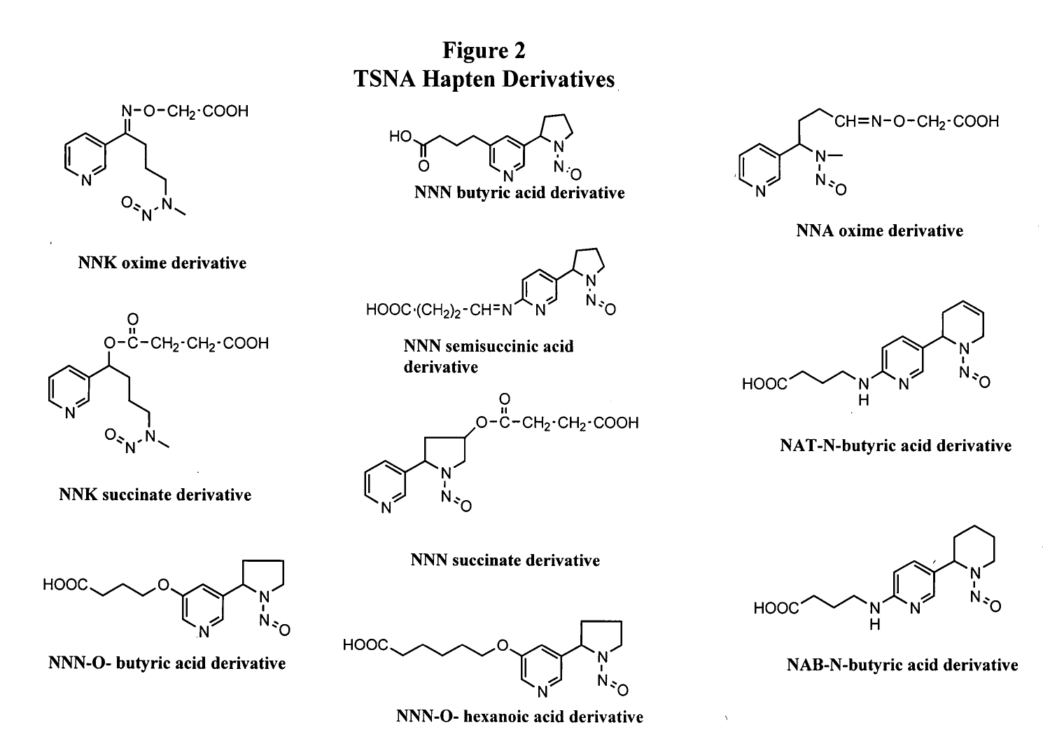

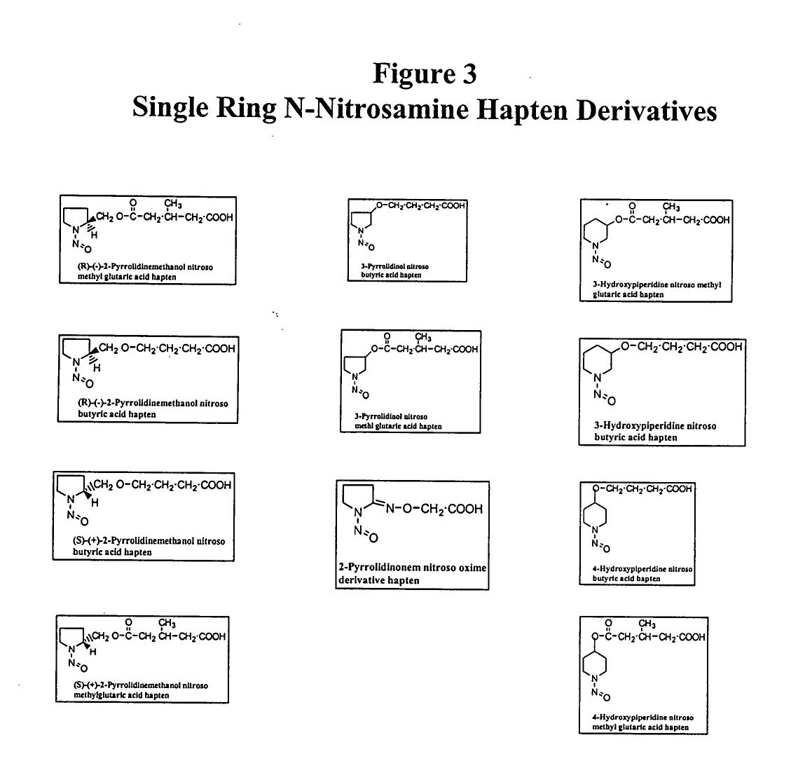

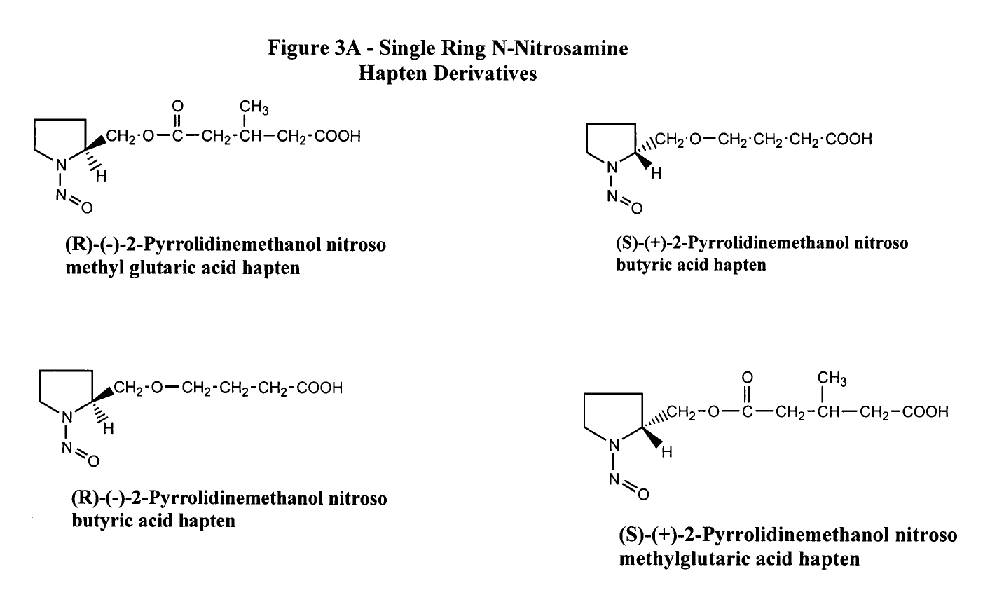

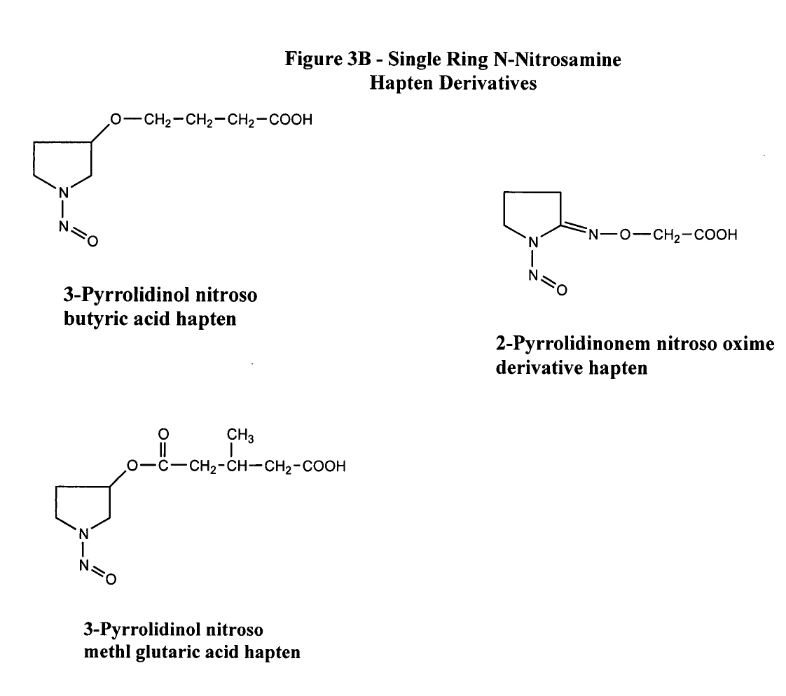

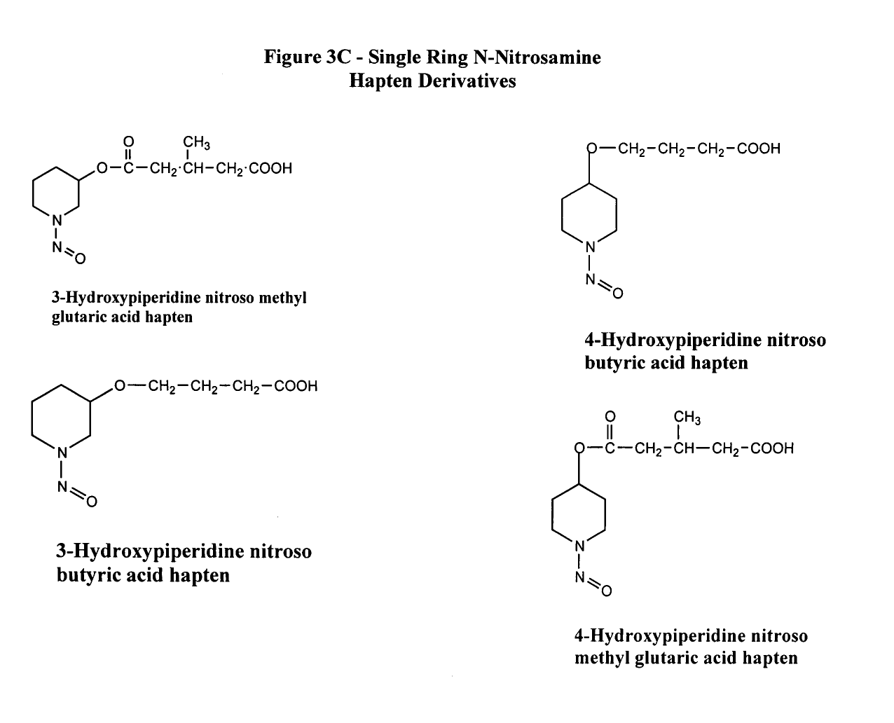

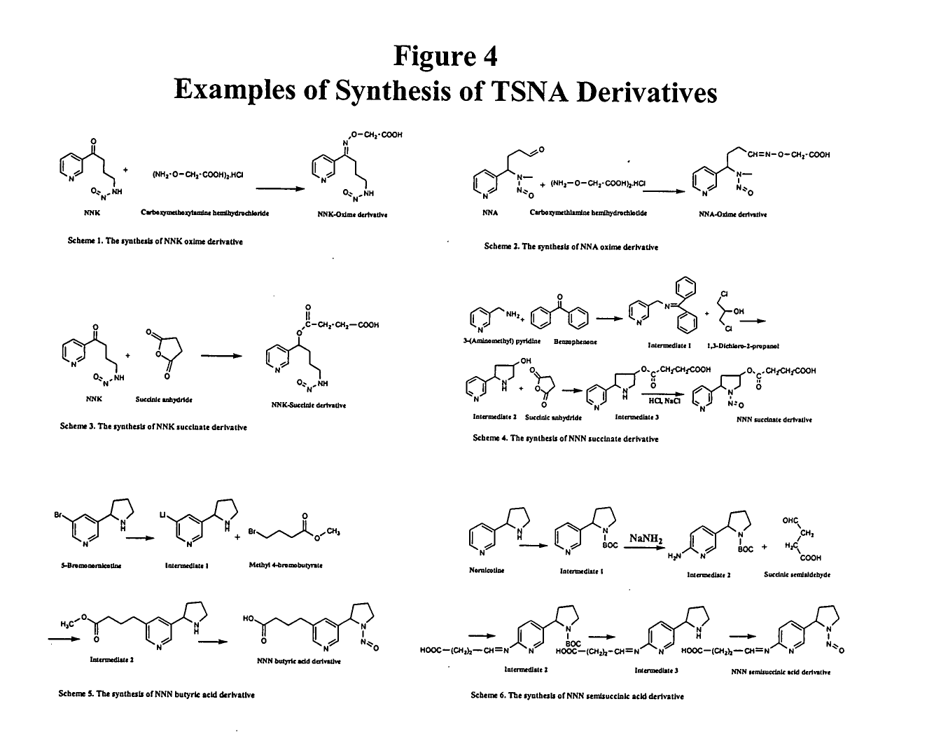

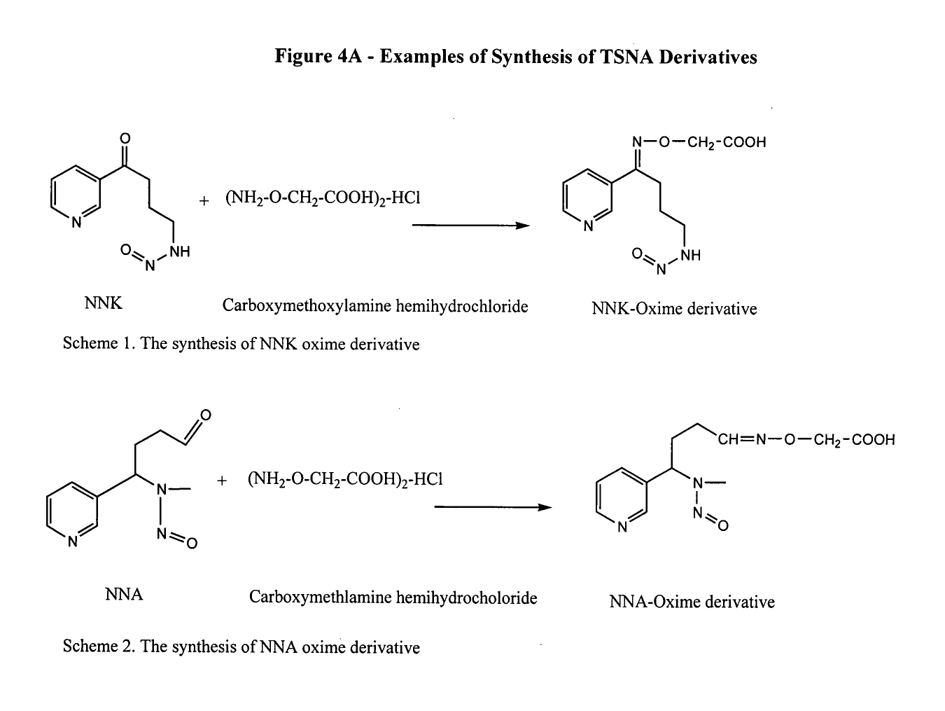

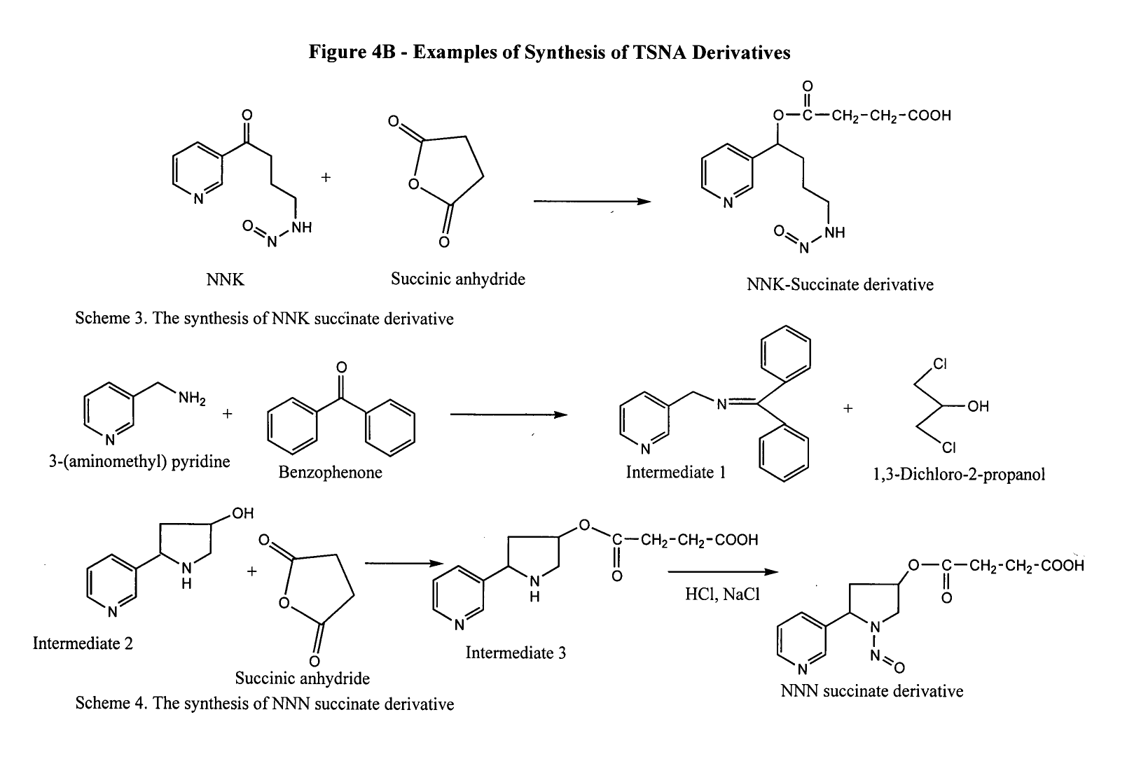

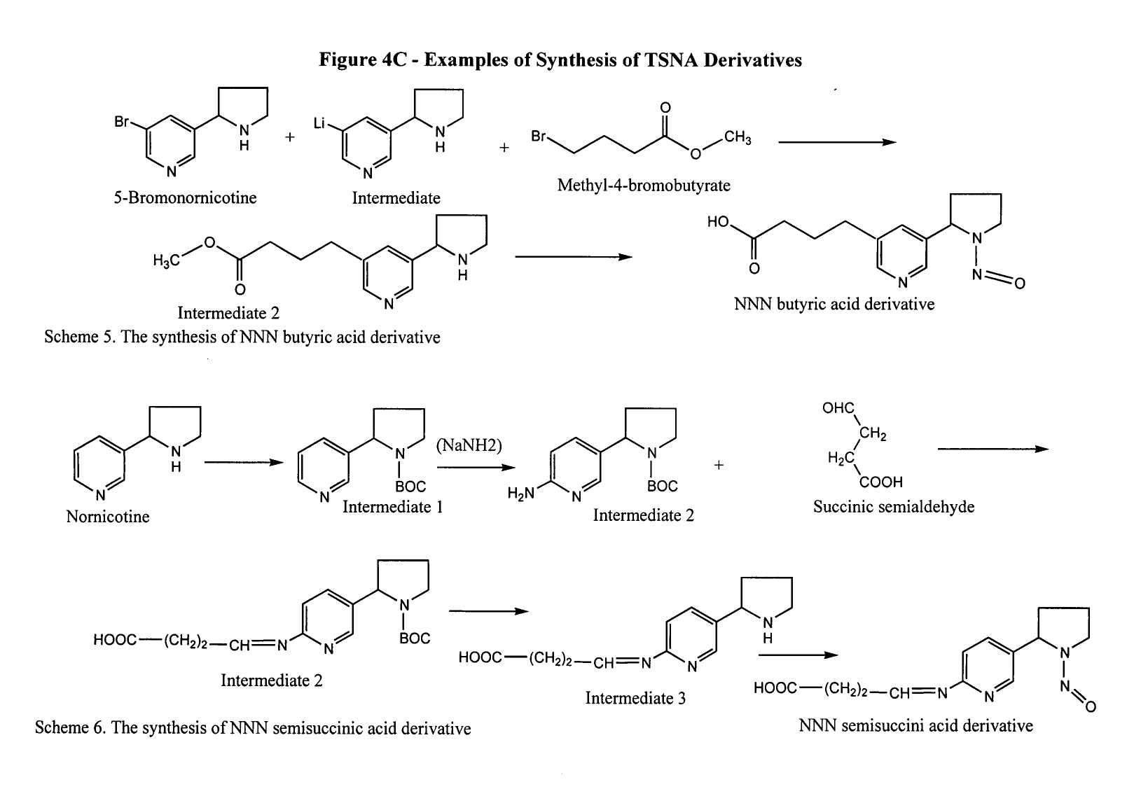

The hapten has or contains a structure that is similar to one or more TSNAs or portions thereof. Examples of haptens include, but are not limited to, derivatized TSNAs and other derivatized N-nitrosamines. Examples of TSNAs are included among the compounds depicted in FIG. 1. In preferred embodiments, derivatization results in the addition of a moiety to which the immunogenic carrier will attach, either directly or indirectly. A preferred hapten is a TSNA derivatized at a site other than the nitroso group, preferably a site that is separated from the nitrosamine by a distance of at least one carbon atom. Another preferred hapten is derivatized at a site on the pyridine (also referred to herein as pyridyl) ring of a TSNA. Haptens are prepared by any means, including but not limited to derivatizing a TSNA or other molecule as that molecule occurs naturally or as a byproduct of tobacco processing, de novo synthesis of a molecule containing the structure of a derivatized TSNA or other derivatized molecule, or combinations thereof. In some embodiments, multivalent haptens containing multiple structures that are similar to TSNAs or portions thereof are used, including, but not limited to multivalent haptens manufactured synthetically. Because of the stability of the pyridine rings of TSNAs and other similar compounds, haptens are optionally synthesized de novo rather than by derivatization of a preexisting TSNA molecule or portion thereof. Examples of derivatized haptens suitable for conjugation are shown in FIG. 2 and FIG. 3. In one preferred embodiment shown in FIG. 2, NNK is derivatized at the keto group. In other embodiments, other locations on the pyridyl ring are substituted. Methods for synthesizing these derivatives are shown in FIG. 4.

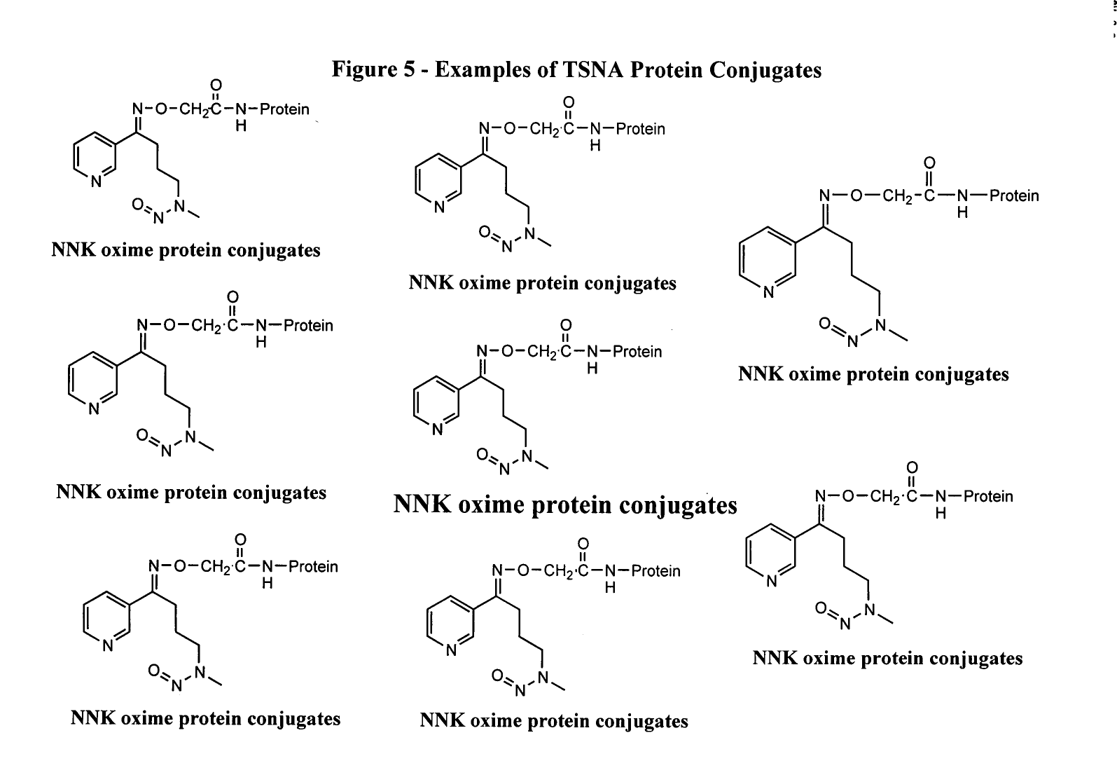

The additional moiety on the hapten, such as the additional moiety added during derivatization, is a linking group that will bond to an immunogenic carrier or is a moiety that will allow formation of a bond with such a linking group. In some embodiments, multiples moieties are used, allowing linkage to multiple carriers. Any linking group that is effective to connect the hapten or, preferably, a derivative on the hapten to the carrier may be used. In some embodiments, the linker has the structure R—(CH₂)ₙ—R₁ wherein n is an integer from 1 to 14 and R and R₁ are moieties that can be reacted to result in formation of a bond with the TSNA and the immunogenic carrier, respectively. Preferred linkers are moieties that will result in the TSNA and carrier molecule being separated by a chain having a length of at least one carbon molecule. Suitable linkers include but are not limited to succinic acid, butyric acid, and hexanoic acid. Preferred examples are succinic acid, butyric acid, and hexanoic acid, which are connected to the TSNA through ester or ether linkages and have a free residue that can be made to react with the immunogenic carrier resulting in the connection of the carrier to the TSNA either directly or indirectly through one or more covalent bonds. An example of such a residue is a carboxylic acid that can react with an amine terminal group of an amino acid using a suitable linking reagent, such as a carbodiimide. Examples of haptens linked to suitable protein carriers are depicted in FIG. 5.

In some embodiments, the hapten is a molecule or substance that differ structurally from a complete TSNA molecule, but that elicits production of antibodies that bind TSNA analytes. Examples of such molecules or substances include, but are not limited to: fragments of one or more TSNAs; synthetic molecules that differ structurally from TSNAs but that contain one or more epitopes or other moieties from TSNAs; and constructs comprising multiple TSNA epitopes. Examples of such molecules include, but are not limited to those in FIG. 3 (depicted as derivatized). It is further contemplated that any N-nitrosamine or a nitroso group added to a secondary amine containing compound coupled as described below may be a suitable hapten.

Any immunogenic carrier effective to cause an immune response to the immunogen may be bound to the hapten. In some embodiments, the immunogenic carrier is a large molecule, such as a protein, having the ability to provoke an immune response when administered to an animal. Suitable immunogenic carriers include proteins, polypeptides or peptides such as albumins, hemocyanins, thyroglobulins and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular weight of at least 1,000 daltons, preferably greater than 10,000 daltons. Carrier molecules often contain a reactive group to facilitate covalent conjugation to the hapten. The carboxylic acid group or amine group of amino acids or the sugar groups of glycoproteins are examples of groups often used in this manner. Carriers lacking such groups are often reacted with an appropriate chemical to produce them. Preferably, an immune response is produced when the immunogen is administered to animals such as mice, rabbits, rats, goats, sheep, guinea pigs, chickens, and other animals, most preferably by the injection mice or rabbits.

Methods of Making Antibodies

The invention further includes methods for making the antibodies described herein. In one embodiment, the antibodies are made by administering the immunogen described above to an animal under conditions effective to cause an immune response, and the collection and purification of the antibodies from a biological fluid such as blood in accordance with methods well known to those skilled in the art. Any method for making any type of antibody, including monoclonal and polyclonal antibodies, may be used. A preferred method produces monoclonal antibodies.

Monoclonal antibodies are generated by any method effective to produce them. One embodiment uses a modified version of the method of Kearney, et al., J. Immunol. 123:1548-1550 (1979), which is incorporated by reference herein. Briefly, animals (for example, mice or rabbits) are inoculated with the immunogen in adjuvant, and spleen cells are harvested and mixed with a myeloma cell line, (for example, P3X63Ag8,653). The cells are induced to fuse, for example by the addition of polyethylene glycol. Hybridomas are chemically selected by plating the cells in a selection medium containing hypoxanthine, aminopterin and thymidine (HAT). Hybridomas producing antibodies are cloned, expanded and stored frozen for future production.

Hybridomas are subsequently screened for the ability to produce monoclonal antibodies of the desired affinity and specificity. Examples of methods for screening an antibody to determine reactivity with a specific hapten may include screening against a hapten coupled to a protein carrier immobilized on a surface. The presence of a signal on the solid surface indicates the existence of an antibody reactive with the hapten. Presence of antibody against the hapten is subsequently detected using, for example, a commercially available enzyme labeled antibody directed against immunoglobulin of the species being used for antibody production. In other embodiments, the antibodies being generated for evaluation are directly or indirectly immobilized onto a solid surface and combined with hapten directly labeled with enzyme. Again, a signal on the solid surface indicates the existence of an antibody reactive with the hapten. Similarly, the ability to detect compounds to inhibit these antibodies is well known and includes the known art of competitive immunoassay. In some embodiments, screening strategies include adding a compound that one does not desire the antibodies to recognize. An example is the addition of nicotine to the screening process such that any antibodies strongly reactive with nicotine will be excluded and appear non reactive to the hapten of interest.

In some screening assays using a competitive format, both antibody and the labeled ligand are titered to achieve an immunoassay that is capable of being inhibited by the presence of analyte in the sample. Additionally, appropriate choice of ligand and the extent to which it is conjugated to the label can influence both specificity and sensitivity of the assay. In one embodiment derivatives of the TNSA used for immunization are also used to prepare the conjugate. In a second embodiment a different TSNA than that used for immunization is used to prepare the conjugate. In some embodiments, antibodies are generated by immunizing an animal with an immunogenic amount of the antigen emulsified in an adjuvant such as Freund's complete adjuvant, administered over a period of weeks in intervals ranging between two weeks and six weeks. In a preferred embodiment, the method includes a first immunization in Freund's complete adjuvant and subsequent immunizations in Freund's incomplete adjuvant (at biweekly to monthly intervals thereafter) then isolating the antibodies from the serum, or fusing spleen from the animal cells to myeloma cells to make hybridomas which express the antibodies in culture. Test bleeds are preferably taken at fourteen day intervals between the second and third immunizations and production bleeds at monthly intervals thereafter.

In some embodiments, conventional hybridoma techniques are employed to prepare monoclonal antibodies (MAbs) for use in the assay of the present invention. The method for producing MAbs is extremely powerful and allows for the preparation of a defined and reproducible antibody reagent. Hybridoma technology permits one to explore the entire antibody producing B-lymphocyte repertoire of the immune system and to select unique antibody producing cells that produce antibodies having unique binding characteristics. The production of polyclonal antisera is much less controlled since polyclonal antisera contain numerous antibody populations each having varying specificity and sensitivity characteristics that are the products of numerous responding B-cell clones. MAb reagents are also homogeneous with a defined specificity, unlike polyclonal antisera which contain a mixed population of antibodies. The use and appropriate selection of hybridoma cell lines provides MAb reagents that offer unique performance characteristics to the test system and consistency of the methods that utilize them.

In some embodiments, hosts responding to the immunization protocol are selected as splenocyte (or lymphoid cell) donors for hybridoma production. A culture of immune lymphocytes fused with modified myeloma cells, using polyethylene glycol (PEG) with modified myeloma cells in a defined tissue culture medium, such as HAT (hypoxanthine, aminopterin, thymidine), is capable of providing a variety of fusion products, such as s-s, s-m, and m-m (with “s” as an abbreviation for splenocyte and “m” as an abbreviation for myeloma cell). Within the tissue culture medium the s-s fusion product normally has a short lifetime and dies within days. Also, the m-m fusion product has a very short lifetime in the tissue culture medium used, lacking the metabolic components needed for DNA synthesis. However the s-m fusion product (or hybridoma) survives in tissue culture and retains the antibody-producing characteristics of the splenocyte parent, and the high rate of growth and relative immortality of the myeloma cell parent. These hybridoma cell lines replicate readily in culture producing daughter cells that provide a reproducible, homogeneous, and consistent supply of the monoclonal antibody of the present invention. Selection of the appropriate cell line provides the monoclonal antibody of a preferred embodiment of the present invention.

Ligand

The ligand described herein includes any molecule that binds to one or more TSNAs. A preferred ligand is an antibody, which binds to or reacts with an analyte such as an antigen. In some embodiments, the ligand is an antibody produced by immunizing an animal with the immunogen described above. In some embodiments, the ligand is a protein or polypeptide with binding affinity for one or more TSNAs. In some embodiments, the protein is an immunoglobulin. In some embodiments, the ligand is an antibody produced by immunizing an animal with an immunogenic composition containing one or more of the immunogens described herein or produced by one of the methods described above.

In some embodiments, the ligand has a high affinity for one or more TSNAs. In other embodiments, the ligand has a high affinity for TSNAs and a minimal, low or nonexistent crossreactivity with nicotine. In one preferred embodiment, reactivity with the TSNA is at least 100 times the reactivity with nicotine. In another preferred embodiment, reactivity with the TSNA is at least 1,000 times the reactivity with nicotine. In another preferred embodiment, reactivity with the TSNA is at least 10,000 times the reactivity with nicotine. In some embodiments, the ligand has a high affinity for multiple TSNAs. In other embodiments, the ligand has a high affinity for one or more TSNAs and has a minimal or low affinity, preferably less than 1%, or a nonexistent or nondetectable crossreactivity with one or more other TSNAs. In one preferred embodiment, the ligand binds to the nitroso group of a TSNA.

In some embodiments, the ligand is labeled to allow detection of analyte in a sample. For example, the labeled ligand is combined with the sample, and the labeled ligand-analyte complex is detected. The ligand is, for example, labeled during ligand production, or a label is conjugated to the ligand by joining it to the ligand, either covalently or non-covalently. A wide variety of labels and conjugation techniques are known and are reported extensively in both the scientific and patent literature. Any label and any conjugation technique may be used. Suitable labels include radioactive molecules, enzymes, substrates, cofactors, inhibitors, fluorescent moieties, chemiluminescent moieties, chromagenic moieties, magnetic particles, and the like. The particular label or detectable group used in the assay is not a critical aspect of the invention. The detectable group can be any material having a detectable physical or chemical or other property. Such detectable labels have been well developed and, in general, any label useful in such methods can be applied to the present method. Thus, examples of labels include any composition detectable by spectroscopic, photochemical, biochemical, immunochemical, electrical, optical or chemical means. Useful labels in the present method include fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., hydrolases, particularly phosphatases, esterases and glycosidases, or oxidoreductases, particularly peroxidases, LacZ, CAT, horseradish peroxidase, alkaline phosphatase and others, commonly used as detectable enzymes, either in an EIA or in an ELISA), and colorimetric labels such as colloidal gold or colored glass or plastic (e.g. polystyrene, polypropylene, latex, etc.) beads. The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non-radioactive labels are often attached by indirect means. In one embodiment, a molecule having a known binding affinity (e.g., biotin) is covalently bound to the molecule. The ligand then binds to another molecule, (e.g., streptavidin), which is either inherently detectable or covalently bound to a signal system, such as a detectable enzyme, a fluorescent compound, or a chemiluminescent compound. Any pair of molecules having such binding affinities that will function as part of this assay can be used. In some embodiments in which the ligand has a natural anti-ligand, for example, biotin, thyroxine, and cortisol, the ligand is used in conjunction with the labeled, naturally occurring anti-ligands. Alternatively, any haptenic or antigenic compound can be used in combination with an antibody.

In some embodiments, the ligands are conjugated directly to signal generating compounds, e.g., by conjugation with an enzyme or fluorophore. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

Antibodies

As mentioned above, a preferred ligand is an antibody. The preferred antibody is a monoclonal antibody, due to its higher specificity for analyte. Preferred antibodies include, but are not limited to, those monoclonal antibodies designated herein as 245B2, 245B4, 245B5, 245B6, 245B8, 245B9, 245B12, 245B13, 245B18, 245B19, 245B42, 245J3, 245J4, 245J7, 245J9, 245J10, 245J12, 245J14, 245J15, 245J16, 245J18, 245J19, 245J23, 245J28, 245J30, and 245J31. Especially preferred antibodies are the monoclonal antibodies designated as 245B5 and 245J15. Antibodies 245B5 and 245J15 are produced by hybridomas that were deposited with the American Type Culture Collection, 10801 University Boulevard, Manassas, Va. 20110-2209 on or before May 28, 2004. Hybridomas producing monoclonal antibodies 245B5 and 245J15 were deposited as ATCC Accession Nos. (Accession Nos. to be provided by depository at a later date), respectively.

Any of the labels discussed above for ligands may be used for antibodies. Some preferred labels for use in immunoassays are generally known to those skilled in the art and include enzymes, radioisotopes, and fluorescent, luminescent and chromogenic substances including colored particles such as colloidal gold and latex beads. Furthermore, in some embodiments, the antibody is labeled indirectly by reaction with labeled substances that have an affinity for immunoglobulin, such as protein A or G or second antibodies. In some embodiments, the antibody is conjugated with a second substance and detected with a labeled third substance having an affinity for the second substance conjugated to the antibody. For example, in one embodiment the antibody is conjugated to biotin and the antibody-biotin conjugate is detected using labeled avidin or streptavidin. In one embodiment, the antibody is conjugated to a hapten and the antibody-hapten conjugate is detected using labeled anti-hapten antibody. These and other methods of labeling antibodies and assay conjugates are well known to those skilled in the art.

Assays

A highly sensitive assay is provided employing a ligand specific for one or more of the TSNA analytes described above. The assay is useful for detecting the presence or amount of one or more TSNAs in one or more samples. A preferred sample is tobacco or a tobacco product or preparation. A preferred assay is an immunoassay that employs an antibody specific for one or more TSNAs, such as the antibodies described above. Another preferred assay uses a non-immunoglobulin protein with binding affinity for one or more TSNAs. The assay provides ligands with affinity and specificity for TSNAs, including, but not limited to, antibodies with specificity to a group of TSNAs, or all TSNAs, and antibodies with specificity for one or more TSNAs while lacking significant crossreactivity for other TSNAs. Preferably, the assays use antibodies having much lower binding affinity, or crossreactivity, with nicotine than with TSNAs.

In embodiments involving immunoassays, the antibodies may be employed in any heterogeneous or homogeneous, sandwich or competitive immunoassay for the detection of one or more TSNAs. Any method or milieu is used to perform the immunoassay, including but not limited to coated tubes, magnetic beads, lateral flow strips, agglutination assays, turbimetric assays and radioimmunoassays. Assays include quantitative assays and qualitative assays, e.g. around a cutoff limit. In some embodiments, assays are read with suitable instrumentation or visually by eye depending on desired accuracy. In some embodiments, the antibody is labeled (directly or indirectly) with a detectable label, coupled to a solid phase, or both. Any method of labeling or coupling may be used. Methods for coupling antibodies to solid phases are well known to those skilled in the art.

In accordance with the immunoassay method, the sample containing or suspected of containing TSNAs is reacted with the antibody for a sufficient amount of time under conditions that promote the binding of antibody to one or more TSNAs in the sample. It will be understood by those skilled in the art that the immunoassay reagents and sample may be reacted in different combinations and orders. A physical means is employed in some embodiments to separate reagents bound to a solid phase from unbound reagents. Examples of such means include, but are not limited to filtration of particles, decantation of reaction solutions from coated tubes or wells, magnetic separation, capillary action, and other means known to those skilled in the art. It will also be understood that a separate washing of the solid phase may be included in the method.

In some embodiments, the ligand is immobilized on a solid phase to facilitate detection. Any solid phase that will allow immobilization may be used. It will be understood by those skilled in the art that examples of solid phases include latex, polystyrene, polyethylene, polypropylene, polycarbonate, nitrocellulose or any other solid material in the shape of test tubes, microtiter plates, beads, microparticles, dip-sticks, test strips, or the like. Other solid phases include, but are not limited to, glass beads, glass test tubes and any other appropriate shape made of glass or plastic. Preferably, the solid phase is a nitrocellulose strip.

After reaction, the existence, concentration, or both of the analyte is determined by the signal generated by a label. The presence, absence, intensity, or location of the signal may be an indicator. In one embodiment the ligand is fixed to a substrate and the sample is contacted with the substrate under conditions effective to cause the ligand to bind analyte in the sample. The bound ligand is also contacted (either subsequently or simultaneously with its contact with the sample) with unbound ligand that is labeled under conditions effective to cause the labeled ligands to bind the analyte that has already bound to the fixed ligand. The substrate is then washed to remove any unbound ligand and the presence and/or concentration of the analyte is indicated by the presence and/or strength of the label signal. In one embodiment, involving a direct assay, the sample is placed under conditions effective to cause any analyte in the sample to become fixed on a substrate. The substrate is then contacted with labeled ligand under conditions effective to cause binding of the ligand to any bound analyte. The substrate is then washed to remove any unbound ligand and the presence and/or concentration of the analyte is indicated by the presence and/or strength of the label signal. In another embodiment, involving a competitive assay, ligands that bind an analyte are bound to a substrate. The sample to be tested is contacted with the substrate together with analyte bound to a label. The substrate is then washed to remove any unbound ligand and the presence and/or concentration of the analyte is indicated by a weakening of the signal as compared to the signal strength when no sample is present. The foregoing are simply examples of assays and any assay method may be used, including other types of direct and indirect assays as well as competitive assays.

It will be understood that the assay is not limited to embodiments in which a label is attached to the ligand. Labels may be used in any part of the assay that will result in a signal that may be interpreted appropriately. In some embodiments, a binding molecule specific for the ligand, such as an antibody, is labeled and the presence of a bound ligand is detected indirectly. In other embodiments, such as some competitive assays, the label is attached to a molecule that competes with analyte in the sample for binding to a ligand. The foregoing are merely examples and should not be considered limiting. In some embodiments, no label is used and presence, location, or concentration of ligand is determined by other means.

Detection of labels may occur by any method. Examples of known methods include, but are not limited to immunoblotting, western analysis, gel-mobility shift assays, fluorescent in situ hybridization analysis (FISH), tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. Any means may be used to detect labels. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels are detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Preferred detection methods include a direct or indirect enzyme-linked immunosorbent assay (ELISA) using a secondary antibody such as a peroxidase-conjugated goat anti-mouse antibody, a competitive ELISA using an immobilized goat anti-mouse antibody, a mouse antibody reactive with the analyte, and an analyte conjugated to a label, or a direct or indirect immunofluorescence assay using a secondary antibody such as a fluorescein isothiocyanate (FITC)-conjugated goat anti-mouse antibody.

In some embodiments, the concentration of one or more TSNAs in the sample is determined either visually and without the need for analytical instrumentation—for example, by comparing the intensity of the color produced by the sample to a standard such as a color card—or by using instrumentation such as a spectrophotometer or reflectometer.

In some embodiments the resulting reaction mixture, or combination of antibody and sample, is prepared in a solution that optimizes antibody-analyte binding kinetics. An appropriate solution is an aqueous solution or buffer. The solution is preferably provided under conditions that will promote specific binding, minimize nonspecific binding, solubilize analyte, stabilize and preserve reagent reactivity, and may contain buffers, detergents, solvents, salts, chelators, proteins, polymers, carbohydrates, sugars, and other substances known to those skilled in the art.

In some embodiments, the ligands provided herein are used to detect targets extracted into solution from a solid material. For example, a solid sample can be extracted with an aqueous liquid, an organic solvent or a critical fluid and the resulting supernatant can be contacted with the ligand. Examples of solid samples include tobacco plants and parts thereof such as leaves, stems and roots, processed tobacco, and tobacco products.

The reaction mixture solution is reacted for a sufficient amount of time to allow the antibody to react and bind to the analyte to form a ligand-analyte complex. The shortest amount of reaction time that results in binding is desired to minimize the time required to complete the assay. One appropriate reaction time period for an immunochromatographic strip test is less than or equal to 20 minutes or between approximately one minute and 20 minutes. A reaction time of less than five minutes is preferred. Most preferably, the reaction time is less than three minutes. By optimizing the reagents, binding may be substantially completed as the reagents are combined.

The reaction is performed at any temperature at which it will occur as desired. This is often the range of temperatures at which the reagents do not degrade or become inactivated. A temperature between approximately 4° C. and 37° C. is preferred. The most preferred reaction temperature is ambient or room temperature (approximately 25° C.).

Strip tests are composed of multiple porous components, membranes and filters through which liquid sample is drawn by capillary action. Analyte in the sample reacts with the test reagents contained within the test strip as it traverses the length of the strip. In one embodiment, in which the goal is to detect an analyte in tobacco, the tobacco is ground into a powder and the analyte extracted from the powder with a liquid that is then separated from the solid material and assayed using the test. The liquid is applied to a chromatographic strip, and the analyte migrates toward the distal end of the strip. As it migrates down the strip, the analyte reacts with reagents applied to or immobilized on the strip causing a detectable signal product. Detection of the signal indicates the presence of the analyte in the sample.

In one preferred embodiment, the assay detects a total concentration of all TSNAs in a sample. In one embodiment, the results of the assay are compared against the results using one or more standards containing known concentrations of total TSNAs, wherein the individual TSNAs are present in relative concentrations that approximate those in tobacco or other materials to be tested. Optionally, multiple standard solutions are used to prepare a calibration curve. Optionally, tests of individual TSNAs are employed as a follow-up test to verify that the proportions are correct, and other proportions are used to develop standards if appropriate for a given sample. Total TSNAs can also be determined based on the results of a quantitative assay for a single TSNA, wherein those results are adjusted based on the known relationship between the concentration of that TSNA and the total TSNA concentration in a tobacco or other material from which the sample is prepared.

A preferred assay is one that is portable. By “portable” it is meant that all materials and equipment necessary to perform the assay weigh less than 50 pounds. A further preferred assay is one whose results can be interpreted visually without the use of analytical instrumentation. An especially preferred assay is portable and produces results that can be interpreted visually without the use of analytical instrumentation.

Assay Kit

An assay kit for the detection of one or more TSNAs in a sample contains one or more of the ligands described above. In some embodiments, the assay kit is an immunoassay kit containing one or more antibodies specific for one or more TSNAs. The kit may additionally contain equipment for obtaining the sample, a vessel for containing the reagents, a timing means, a buffer for diluting the sample, a colorimeter, reflectometer, or standard against which a color change may be measured or a combination thereof. In one embodiment, the antibody is collectively assembled in a kit with conventional immunoassay reagents for detection of TSNAs. The kit may optionally contain both monoclonal and polyclonal antibodies and a standard for the determination of the presence and/or concentration of one TSNA, or a group of TSNAs. In one preferred embodiment, the kit detects a total concentration of all TSNAs in a sample. The kit containing these reagents provides for simple, rapid, on site detection, and, optionally, quantification, of one or more TSNAs.

In a preferred embodiment, the reagents, including an antibody, are dry or lyophilized, preferably on a chromatographic test strip. Addition of aqueous sample to the strip results in solubilization of the dry reagent, causing it to react.

A preferred assay kit is one portable, as that term is defined above. A further preferred assay kit is one whose results can be interpreted visually without the use of analytical equipment. An especially preferred assay kit is portable and produces results that can be interpreted visually without the use of analytical equipment.

The invention will be described in greater detail by way of specific examples. The following examples are offered for illustrative purposes, and are intended neither to limit nor define the invention in any manner.

### EXAMPLE 1

### Preparation of NNK Conjugates

Materials were prepared by immunization as follows: 24.5 mg of NNK (Toronto Research Chemicals, Toronto, Canada) was reacted with a two fold molar excess (51.7 mg) of carboxymethoxylamine hemihydrochloride in 5 ml of pyridine:methanol:water at 1:4:1 for 24 hours in an oil bath at 50-55° C. The reaction mixture was dried by blowing nitrogen through the vessel containing the mixture. Dried reaction mixture was extracted by combining it with methylene chloride (20 ml) and 1 N HCl (20 ml). A second extraction was conducted by combining the aqueous phase from the previous extraction with methylene chloride. A third extraction was performed using the aqueous phase from the second extraction with methylene chloride. Organic phases from all three extractions were pooled and dried to yield 16.8 mg of NNK oxime derivative.

A N-hydroxysuccinimidyl ester was prepared by reacting 16.8 mg of the NNK oxime derivative with a 67.9 mg of 1-Ethyl-3-(3-dimethylaminopropyl) carbodiimide HCl and 40.7 mg of N-hydroxysuccinimide in 2 ml N,N-dimethylformamide for 24 hours at room temperature. The resulting active ester was stored at −20° C.

To prepare haptens conjugated to proteins, 10 mg of BSA or KLH was dissolved in 2 ml of 0.1 M carbonate buffer (0.9 M sodium chloride, pH 9.6) and 0.152 ml of the solution containing the NHS ester of NNK at 0.000059 mMoles/μl added. The reaction was allowed to proceed at room temperature for 24 hours after which non-reacted hapten was removed by dialysis against PBS. Similar reactions were carried out with ovalbumin for screening assays and horseradish peroxidase in connection with assays. For the peroxidase conjugates, varying molar ratios of protein to hapten (1:4, 1:8, and 1:16) were prepared, all of which showed reactivity in the final assay. Peroxidase conjugates were stored in 50% glycerol at −20° C. All other conjugates were stored at 4° C.

### EXAMPLE 2

### Preparation of Monoclonal Antibodies to NNK

Monoclonal antibodies (MAbs) were generated by a modified version of the method of Kearney et al., J. Immunol. 123:1548-1558 (1979). An NNK-oxime derivative hapten was conjugated to either bovine serum albumin or keyhole limpet hemocyanin as described above in Example 1. These two different conjugates were used as immunogens for the immunization of mice. The conjugates were diluted in complete Freund's adjuvant to prepare a 1:1 solution of conjugate:adjuvant.

Swiss Webster mice (approximately eight weeks old) were immunized by injection with the suspension. Mice received two booster injections of the same conjugate/adjuvant at one month and two months after initial injection. At three months after initial injection, spleen cells were harvested from the mice and mixed with myeloma cell line P3X63Ag8,653. The cells were induced to fuse by the addition of polyethylene glycol. Hybridomas were chemically selected by plating the cells in a selection medium containing hypoxanthine, aminopterin and thymidine (HAT).

Hybridomas were subsequently screened for the ability to produce anti-TSNA monoclonal antibodies as determined by a direct bind ELISA using microplates coated with the TSNA derivatives coupled to ovalbumen. Separate screenings were performed using immunogens based upon NNK, NNN, NAT, and NAB. Each immunogen shown in FIG. 3 was screened separately. For screening, microtiter plates (Nunc, Maxisorp) were coated by placing 100 microliters (μl) per well of 0.1 molar carbonate buffer (pH 9.6) containing ovalbumen conjugated to the TSNA being tested at a concentration of 5.0 μg/ml (micrograms per milliliter), and allowing the solution to incubate overnight. The next day the contents of the wells were discarded and unreacted sites on the plastic wells were blocked with 300 μl per well of PCT (1% Casein (Sigma Chemical Co., St Louis, Mo.), 0.05% Tween 20 (Sigma Chemical Co.) in phosphate buffered saline (PBS)) for one hour. Contents were again discarded and 10 μl of culture supernatant from hybridomas being screened were added per well along with 90 μl of PCT. Wells were incubated one hour at room temperature. Contents of the wells were again discarded and wells washed three times with PBS with 0.05% Tween (PT). A 100 μl aliquot of peroxidase-conjugated Rabbit anti Mouse IgG (Catalog # 61-6020, Zymed Corporation, San Francisco, Calif.) in PCT was added to the wells and again incubated for one hour at room temperature. Wells were washed six times with PT and excess liquid removed by tapping. A 100 μl aliquot of a peroxide/3,3′,5,5′-tetramethylbenzidine substrate/chromagen (Catalog# TMBUS, Moss Corporation, Pasadena, Md.) was added. Color was read at 650 nm after fifteen minutes in a microplate reader (SpectraMax Plus, Molecular Devices, Sunnyvale, Calif.).

In this assay, horseradish peroxidase, coupled to the rabbit anti-mouse IgG reduced hydrogen peroxide in the substrate chromagen mixture which, in turn, oxidized the chromagen, creating a blue color. Presence of antibody directed against the hapten of interest immobilized onto the well resulted in an increase in blue color above the background.

Using known techniques, hybridomas producing antibodies were cloned, expanded and stored frozen for future production. MAbs identified included 245B2, 245B4, 245B5, 245B6, 245B8, 245B9, 245B12, 245B13, 245B18, 245B19, 245B42, 245J3, 245J4, 245J7, 245J9, 245J10, 245J12, 245J14, 245J15, 245J16, 245J18, 245J19, 245J23, 245J28, 245J30, 245J31. MAb 245B5 was selected for further analysis because of its preferential reactivity with NNK. MAb 245J15 was selected for further analysis because of its reactivity with several of the TSNAs (NNK, NNN, NAT), with weak reactivity with NAB.

### EXAMPLE 3

### Competitive ELISA Immunoassay

Microtiter plates (Nunc, Maxisorp) were coated by adding 100 μl per well of goat anti mouse IgG Fc specific (Jackson Immunoresearch, West Grove, Pa.) at 2.5 micrograms per milliliter (μg/ml) in phosphate buffered saline (Sigma Chemical Co.) and allowing the solution to sit overnight. The next day, contents were discarded and unreacted sites on the plastic wells were blocked with 300 μl per well of PBT (1% Bovine Serum Albumen (Sigma Chemical Co.), 0.05% Tween 20 (Sigma Chemical Co.) in PBS) for one hour. Contents were again discarded and 100 μl was added per well of a 1/10,000 dilution of hybridoma culture supernatant of monoclonal antibody 245B5. Wells were then incubated one hour at room temperature. Contents of the wells were again discarded and wells washed three times with PBS with 0.05% Tween (PT). Aliquots containing 50 μl of test compounds diluted in PBT were added to the wells in duplicate, followed in less than five minutes by 50 μl of NNK conjugated to horseradish peroxidase (5 μg of horseradish peroxidase/ml). Wells were incubated for one hour at room temperature, after which they were washed six times with PT and excess liquid removed by tapping. A 100 μl aliquot of a peroxide/3,3′,5,5′-tetramethylbenzidine substrate/chromagen (Catalog # TMBUS, Moss Corporation, Pasadena, Md.) was added. Color was read at 650 nm after fifteen minutes in a microplate reader (SpectraMax Plus, Molecular Devices, Sunnyvale, Calif.). The presence of a TSNA reactive with monoclonal antibody 245B5 caused a decrease in the strength of the color signal.

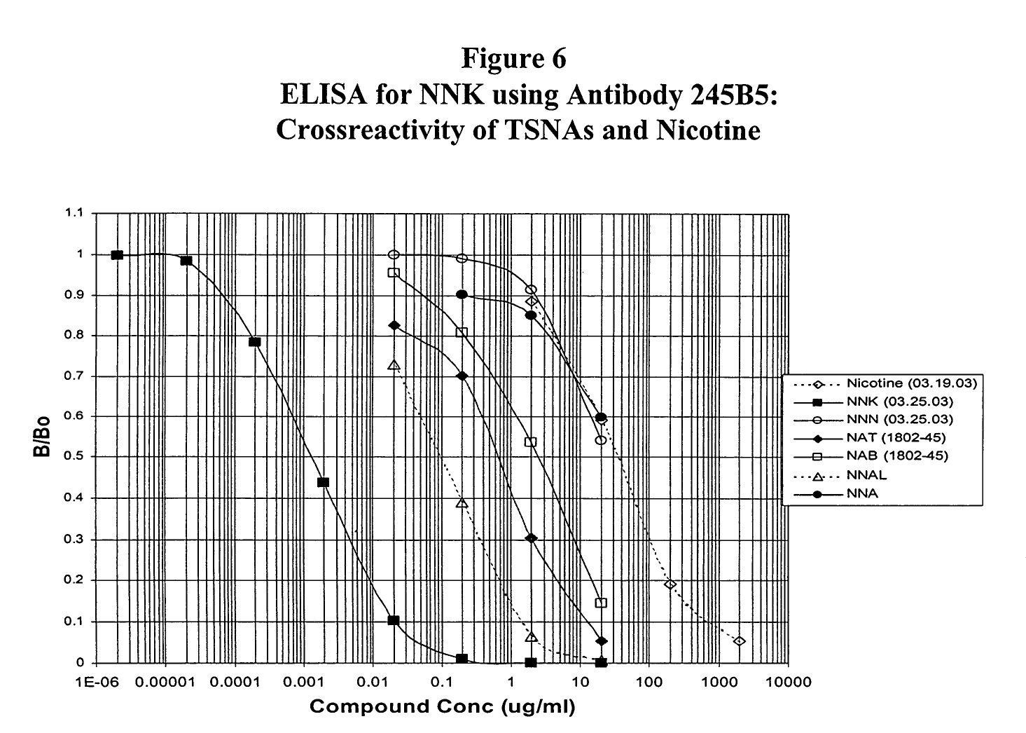

The foregoing procedures were performed with monoclonal antibody 245B5 using the following as test compounds: nicotine, NNN, NAT, NAB, NNAL, NNA, methylanabasine, anatabine, nornicotine, anabasine, and other compounds. FIG. 6 shows the results for nicotine, NNN, NAT, NAB, NNAL, NNA and NNK. TSNAs were purchased from Toronto Research Chemicals. Nicotine and other related compounds were purchased from Sigma-Aldrich, Milwaukee, Wis. The x-axis shows the concentration of the compounds, in micrograms per milliliter. The y-axis is B/Bo, which is the amount of color at 650 nm obtained in the presence of the compound divided by the amount of color obtained when no competing compound is present.

Results of this assay for all compounds are shown in Table 1, where results are shown based on the amount of compound required to give 50% inhibition (as determined by 0.5 B/Bo). Percent crossreactivity is defined as the amount of NNK required to give 50% inhibition divided by the amount of the test compound required to give 50% inhibition, then multiplied by 100. All of the characteristics of this antibody make it well suited for use in analysis of NNK.

TABLE 1
ELISA for NNK using Antibody 245B5: Crossreactivity of
TSNAs and Nicotine

| | Vendor & | % | 50% Inhibition at: | |
|---|---|---|---|---|
| Compound | Catalog No. | Crossreactivity | Ppm | M |
| NNK | TRC* | 100.00 | 0.00135 | 6.5E−09 |
| | M325750 | | | |
| NNAL | TRC M325755 | 1.95 | 0.06923 | 3.3E−07 |
| NAT | TRC N524750 | 0.39 | 0.35 | 1.8E−06 |
| NAB | TRC N524250 | 0.10 | 1.35 | 7.1E−06 |
| NNN | TRC N535000 | 0.0088 | 15.43 | 8.7E−05 |
| Nicotine | Aldrich 186376 | 0.0080 | 16.88 | 1.0E−04 |
| NNA | TRC O860000 | 0.0075 | 18.00 | 8.7E−05 |
| Methyl | TRC M287500 | 0.0005 | 271.90 | 1.6E−03 |
| Anabasine | | | | |
| Anatabine | TRC A637500 | 0.0004 | 311.06 | 1.9E−03 |
| Nornicotine | TRC N757000 | <0.0002 | >200 | >1E−03 |
| Anabasine | TRC A637175 | <0.0002 | >200 | >1E−03 |

Principal TSNAs NNN, NAT, and NAB show well under 1% crossreactivity.
NNAL, the reduced form of NNK shows only 2% crossreactivity.
Nicotine shows only 0.008% crossreactivity.

### EXAMPLE 4

### Immunoassay Capable of Detecting Numerous TSNAs

A total TSNA assay was developed for monoclonal antibody 245J15.

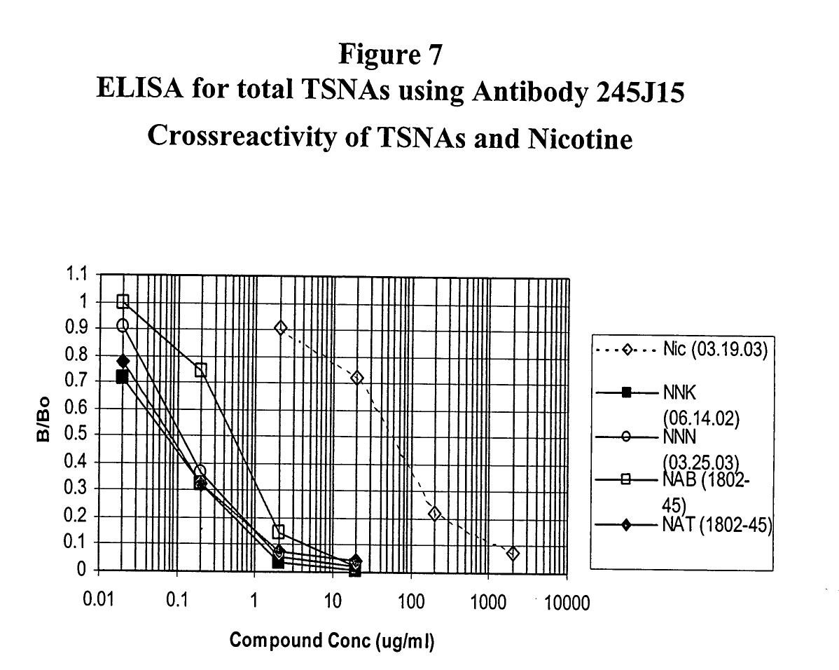

Immunoassays using this antibody and the procedures of Example 3 were conducted for nicotine, NNK, NNN, NAB, NAT, and all other analytes listed in Table 2. Each analyte was obtained from the source identified in Example 3. Results are presented in FIG. 7, with B/Bo having the same meaning set forth in Example 3. Additional results are presented in Table 2.

TABLE 2
ELISA for total TSNAs using Antibody 245J15: Crossreactivity of TSNAs,
Nicotine, and other Compounds

| | Vendor & | | 50% Inhibition at: | |
|---|---|---|---|---|
| Compound | Catalog No. | % Crossreactivity | ppm | M |
| NNK | TRC* M325750 | 100 | 0.06 | 2.90E−07 |
| NNAL | TRC M325755 | 129.6 | 0.05 | 2.20E−07 |
| NAT | TRC N524750 | 46.3 | 0.13 | 6.80E−07 |
| NNN | TRC N535000 | 37.1 | 0.16 | 9.10E−07 |
| NNA | TRC O860000 | 10.8 | 0.56 | 2.70E−06 |
| NAB | TRC N524250 | 8.3 | 0.72 | 3.80E−06 |
| Anatabine | TRC A637500 | 0.7 | 8.3 | 5.20E−05 |
| Methyl Anabasine | TRC M287500 | 0.2 | 30.2 | 1.80E−04 |
| Nicotine | Aldrich 186376 | 0.074 | 80.9 | 5.00E−04 |
| 1-Methyl-3- | Aldrich M79506 | 0.065 | 92.7 | 9.20E−04 |
| pyrrolidinol | | | | |
| Nicotine (Acros) | Acros 18142100 | 0.034 | 178.9 | 1.10E−03 |
| Anabasine | TRC A637175 | 0.028 | 214.9 | 1.30E−03 |
| Nornicotine | TRC N757000 | 0.027 | 223.1 | 1.50E−03 |
| 1-Methyl-2- | Aldrich 155241 | 0.008 | 781.4 | 6.00E−03 |
| piperidinemethanol | | | | |
| Pyridine | Aldrich 360570 | 0.006 | 946.9 | 1.20E−02 |
| (S)-(+)-2- | Aldrich 186511 | 0.006 | 996.7 | 9.90E−03 |
| Pyrrolidine-methanol | | | | |
| 1-Methyl-2- | Aldrich 139513 | 0.005 | 1264.4 | 9.80E−03 |
| pyrrolidineethanol | | | | |
| Piperidine | Aldrich 104094 | 0.005 | 1300.3 | 1.50E−02 |
| (R)-(−)-2- | Aldrich 281697 | 0.003 | 1898.1 | 1.90E−02 |
| Pyrrolidine-methanol | | | | |
| 2-Pyrrolidinone | Aldrich P74370 | 0.002 | 2594 | 3.00E−02 |
| 3-Pyrrolidinol | Aldrich P74354 | 0.001 | 5373.9 | 6.20E−02 |
| 4-Hydroxypiperidine | Aldrich 391468 | 0 | 12282.5 | 8.90E−02 |
| hydrochloride | | | | |
| DL-Pipecolinic Acid | Aldrich P45850 | 0 | 13630.2 | 1.10E−01 |
| 3-Hydroxypiperidine | Aldrich 174416 | 0 | 17492.7 | 1.30E−01 |
| hydrochloride | | | | |
| Nipecotic Acid | Aldrich 211672 | 0 | 24193.5 | 1.90E−01 |

*TRC = Toronto Research Chemicals

Again, percent crossreactivity is defined as the amount of NNK required to give 50% inhibition divided by the amount of the test compound required to give 50% inhibition, then multiplied by 100.

### EXAMPLE 5

### Total TSNA Immunoassay

An immunoassay using antibody 245J15 is used to detect total TSNA levels in tobacco. A suitable standard for such an assay is a mixture of NNN, NAT, NNK, and NAB in the approximate proportions they occur in tobacco (roughly 1:1:0.3:0.05). A solution in water is prepared containing 0.213 μg/ml of NNN, 0.213 μg/ml of NAT, 0.064 μg/ml of NNK, and 0.011 μg/ml of NAB to yield a total TSNA level of 0.5 μg/ml. This solution is used as a standard for the assay described below.

A one gram sample of tobacco or tobacco product is macerated and extracted into 10 ml of a suitable aqueous extraction media. Examples of suitable extraction media include, but are not limited to water, 1 M HCl, 1 M Acetic Acid, citrate phosphate buffer at pH 2.5, and 1% triethanolamine. A sample of the extract is then tested, relative to a sample of a standard or control. In this example, a tobacco sample that had equal activity in the assay compared to the standard of 0.5 μg/ml has (0.5 μg/ml×10 ml extract/1 gm tobacco) 5 μg of TSNA per 1 gram of tobacco. A sample with more color in the assay (less inhibition of conjugate binding than the standard) has less than 5 μg TNSA per 1 gram of tobacco. A sample with less color (greater inhibition of conjugate binding than the standard) has greater than 5 μg TNSA per 1 gram of tobacco.

Optionally, standards having other concentration of total TSNAs are used to create other cutoff limits as well as levels of standards used to generate a curve from which quantitative values can be obtained.

### EXAMPLE 6

### Polyclonal Assay for NNK

NNK oxime derivatives were prepared and conjugated to KLH and BSA as described in Example 1. The conjugates were diluted in complete Freund's adjuvant to prepare a 1:1 solution of conjugate:adjuvant. New Zealand White Rabbits (6 to 12 months of age) were immunized by injection with the suspension. Rabbits received two booster injections of the same conjugate/adjuvant at one month and two months after initial injection. At three months time, point test bleeds were tested against microtiter plates coated with oxime-derivatized NNK prepared and conjugated to ovalbumen using procedures described in Example 1. Eight of eight rabbits immunized with the NNK oxime derivatives produced antibody that was reactive with ovalbumen to which NNK oxime had been coupled.

All of these rabbits were used individually to produce competitive assays, as described in Example 3, with the exceptions that a 1:250,000 dilution of antisera in PBT was substituted for the supernatant and the microtiter plates were first coated with goat anti rabbit IgG (Catalog # 115-005-008, Jackson Immunoresearch, West Grove, Pa.) rather than goat anti mouse IgG. All other details were the same. For one such rabbit, sensitivity of this assay at the 50% inhibition point was 0.1 μg NNK per ml. Crossreactivity at the 50% point, as previously defined for NNN, NAT, NAB, and nicotine was 1.5%, 1.7%, 1.6% and 0.024% respectively. Other rabbits showed roughly comparable sensitivity and specificity.

### EXAMPLE 7

### Contribution of the N-Nitroso group to immunoreactivity

The affinity of monoclonal antibodies 245B5 and 245J15 to the N-nitroso group on compounds was evaluated using the immunoassay of Example 3 and the immunoassay of Example 4. Parent compounds lacking nitroso groups but having secondary amine groups were run in the immunoassays as previously described. Identical immunoassays were also conducted for compounds that were identical with the parent compounds except that for the addition of the nitroso group to the secondary amine in the compounds. NNN, NAT, and NAB are all examples of TSNAs. N-nitrosopyrollidine may be found in tobacco smoke, but is not a TSNA. N-nitrosodibutylamine is an example of a nitrosamine not routinely found in tobacco or tobacco smoke. Results for the immunoassay using antibody 245B5 are shown in Table 3, where results are based on the molar concentration of compound required to give 50% inhibition (as determined by B/Bo) of the immunoassay. In all instances a significant increase in reactivity with monoclonal antibody 245B5 was observed with the addition of the nitroso group.

TABLE 3
Effect of N-Nitroso group on ELISA using Antibody 245B5

| | | | 50% |
|---|---|---|---|
| | 50% Inhibition | | Inhibition |
| Parent compound | at (M) | N-Nitroso derivative | at (M) |
| Nornicotine | >1E−03 | NNN | 8.7E−05 |
| Anatabine | 1.90E−03 | NAT | 1.8E−06 |
| Anabasine | >1E−03 | NAB | 7.1E−06 |
| Pyrrolidine | 2.0E−02 | N-Nitrosopyrrolidine | 3.0E−03 |
| Dibutylamine | >5.0E−02 | N-Nitrosodibutylamine | 1.6E−02 |

Results for the immunoassay using antibody 245J15 are shown in Table 4, where results are based on the molar concentration of compound required to give 50% inhibition (as determined by B/Bo) of the immunoassay. In all instances introduction of the nitroso group significantly increased the reactivity in the immunoassays suggesting a critical role for the nitroso group in antibody reactivity.

TABLE 4
Effect of N-Nitroso group on ELISA using Antibody 245J15

| | | | 50% |
|---|---|---|---|
| | 50% Inhibition | | Inhibition |
| Parent compound | at (M) | N-Nitroso derivative | at (M) |
| Nornicotine | 1.5E−03 | NNN | 9.1E−07 |
| Anatabine | 5.2E−05 | NAT | 6.8E−07 |
| Anabasine | 1.3E−03 | NAB | 3.8E−06 |
| Pyrrolidine | 2.0E−02 | N-Nitrosopyrrolidine | 5.0E−03 |
| Dibutylamine | >5.0E−02 | N-Nitrosodibutylamine | 1.00E−02 |

### EXAMPLE 8

### Preparation of a Lateral Flow Immunochromatography Assay

NNK oxime is conjugated to bovine immunoglobulin G (bovine IgG) using the methods of Example 1. This hapten protein conjugate is sprayed at 0.1 mg/mL in PBS onto a nitrocellulose membrane (Millipore, Bedford, Mass. Cat No. HF07054500) using a Biodot XYZ3000-dispensing platform sprayer (Irvine, Calif.) to form a test line. Goat anti-mouse IgG (Lampire Biological Labs, Pipersville, Pa.) is sprayed at 1 mg/mL in PBS to form a control line. Test and control lines are positioned on the membrane such that the test line will be encountered by sample and gold prior to the control line. Monoclonal antibody 245J15 is conjugated to colloidal gold (BBI, Cardiff, UK; 40 nm) using standard methods (Beesley, J. E. (1989). COLLOIDAL GOLD: A NEW PERSPECTIVE FOR CYTOCHEMICAL MARKING. Royal Microscopical Society Handbook No 17, Oxford University Press, New York) and dried onto polyester pads (Reemay 2033, Ahlstrom, Mt. Holly Springs, Pa.). Sprayed nitrocellulose membrane and monoclonal antibody-gold-treated polyester pads are laminated onto plastic backing. A sample filter paper is placed below the gold pad at the sample application end of the strip. A wicking paper is placed above the membrane to facilitate continuous capillary flow. The assembly is cut into test strips using a guillotine cutter.

For sample analysis, 500 μl of liquid sample containing an extract of tobacco as previously described in Example 5 is placed into a 1.8 mL microcentrifuge tube. The test strip is placed into the vial where only the sample filter pad contracted the sample. The test strip is allowed to develop in the sample for ten minutes. Following ten minutes, the test strip is removed from the sample and the results are interpreted. If the test line is of the same or greater color intensity as the control line, the sample has less than 5 μg of TSNA per gram of tobacco. If the test line is of lesser color intensity as the control line, the sample has equal to or more than 5 μg of TSNA per gram of tobacco. Optionally, other extraction schemes and other concentrations are employed to achieve other desired cut-off limits. Such assays are run in a laboratory or non laboratory (on site) setting.

Alternatively, monoclonal antibody 245J15 is sprayed at 0.05 mg/mL in PBS onto nitrocellulose membrane to form a test line. Goat anti-bovine IgG (Lampire Biological Labs, Pipersville, Pa.) is sprayed at 1 mg/mL in PBS to form a control line. Hapten protein conjugate of NNK-oxime bovine IgG is conjugated to 40 nm colloidal gold. All other details of strip construction, running of the test and interpretation of results are as described above.

All patents, publications and abstracts cited above are incorporated herein by reference in their entirety. It should be understood that the foregoing relates only to preferred embodiments of the present invention and that numerous modifications or alterations can be made therein without departing from the spirit and the scope of the present invention as defined in the following claims.

1. A composition comprising a ligand, wherein the ligand has a binding affinity for one or more tobacco-specific nitrosamines.

2. The composition of claim 1, wherein the ligand comprises an antibody having a binding affinity for one or more tobacco-specific nitrosamines.

3. The composition of claim 2, wherein the antibody has a binding affinity for one or more tobacco-specific nitrosamines that is at least 100 times greater than its binding affinity for nicotine.

4 The composition of claim 2, wherein the antibody has a binding affinity for one or more tobacco-specific nitrosamines that is at least 1,000 times greater than its binding affinity for nicotine.

5. The composition of claim 2, wherein the antibody has a binding affinity for one or more tobacco-specific nitrosamines that is at least 10,000 times greater than its binding affinity for nicotine.

6. The composition of claim 2, wherein the antibody has a significantly higher binding affinity for one or more tobacco-specific nitrosamines than its binding affinity for other tobacco-specific nitrosamines.

7. The composition of claim 2, wherein the antibody has a binding affinity for all tobacco-specific nitrosamines.

8. The composition of claim 2, wherein the antibody has a binding affinity for N-nitroso groups of tobacco-specific nitrosamines.

9. The composition of claim 2, wherein the antibody is 245J31.

10. The composition of claim 2, wherein the antibody is 245B5.

11. An immunogen comprising a derivatized tobacco-specific nitrosamine conjugated to an immunogenic carrier.

12. A method for making antibodies having binding affinities to one or more tobacco-specific nitrosamines, comprising administering to an animal an immunogen, wherein the immunogen comprises a derivatized tobacco-specific nitrosamine conjugated to an immunogenic carrier, in such an amount and under such conditions as to cause an immune response to the immunogen.

13. A method for detecting one or more tobacco-specific nitrosamines in a sample, the method comprising:
reacting the sample with a ligand, wherein the ligand has a binding affinity for one or more tobacco-specific nitrosamines, for a time and under conditions sufficient to form a complex between the ligand and the tobacco-specific nitrosamines; and
detecting the complex either directly or indirectly as a measure of the presence of one or more tobacco-specific nitrosamines in the sample.

14. The method of claim 13, wherein the sample contains tobacco.

15. The method of claim 13, wherein the complex is detected visually without the aid of analytical instrumentation.

16. The method of claim 13, wherein the ligand is labeled.

17. The method of 16, wherein the label is selected from the group consisting of a radioactive molecule, enzyme, substrate, cofactor, inhibitor, fluorescent moiety, chemiluminescent moiety, chromagenic moiety, magnetic particle, and the like.

18. The method of claim 13, wherein the ligand is directly or indirectly immobilized onto a solid phase.

19. A kit for detecting one or more tobacco-specific nitrosamines in a sample, comprising an antibody possessing a binding affinity for one or more tobacco-specific nitrosamines.

20. The kit of claim 19, wherein the ligand is directly or indirectly immobilized onto a solid phase.
